(19) United States
(12) Patent Application Publication
(10) Pub. No.: US 2008/0021278 A1
(43) Pub. Date: Jan. 24, 2008

(54) Surgical device with removable end effector

(76) Inventors: Robert F. Leonard, Atlanta, GA (US); Scott Kester, Snellville, GA (US); David J. Booth, Lawrenceville, GA (US); David Feng, Mason, OH (US)

Correspondence Address: ALLEGIANCE CORPORATION, 1430 WAUKEGAN ROAD, MKGAW PARK, IL, 60085, US

(21) Appl. No.: 11/724,041

(22) Filed: Mar. 14, 2007

Related U.S. Application Data
(63) Continuation of application No. 11/492,688, filed on Jul. 24, 2006, now abandoned.

(51) Int. Cl. A61B 1/313

(52) U.S. Cl. 600/129

(57) ABSTRACT

A surgical device and end effector connection system therefor include an easy-to-engage mechanism for connecting a handle/shaft/actuation rod unit to an end effector. This mechanism may utilize an axially engageable inner ball/socket inner connection with a threaded outer connection, or an inner bayonet connection with either an outer bayonet connection or an outer threaded connection. In each of the embodiments, an outer portion of the end effector preferably is statically engaged with the shaft, and an inner portion of the end effector is engaged with the actuation rod such that axial movement of the actuation rod can actuate a tool end of the end effector. The present invention further relates to methods of use for the laparoscopy device.

### FIELD OF THE INVENTION

The present invention relates generally to medical devices, and more specifically to a surgical device useful in laparoscopic procedures and having a removable end effector, as well as methods of use for the device.

### BACKGROUND

A wide variety of instruments are presently known and employed in the art of laparoscopic surgery, which typically involves gaining access to a patient's peritoneal cavity by way of an endoscope and/or trocars passing through the patient's abdominal wall. Many such medical instruments include an operable end effector in the form of cutting blades, forceps or other devices wherein the device is passed through a discrete opening providing access to the patient's interior. The opening may be surgically created (e.g. using a trocar) or naturally occurring (e.g., via the anterior or posterior openings of the alimentary canal). In such devices the surgeon must be able to manipulate adequately a distal, internally disposed end of the laparoscopic instrument to perform the required surgical routine. For this reason, laparoscopic devices are often designed such that a movable rod extending coaxially through a distally-projecting elongate shaft may be manipulated from the proximal, operative end of the instrument to manipulate a distal end effector of the instrument (e.g., forceps, scissor-type blades or the like).

It is well known that in many such laparoscopic instruments, such as those employing cutting blades, it is periodically necessary to replace the blades by new and sharper blades, in which case it is often economically expedient merely to remove the tip of the instrument and replace it with a new tip, rather than discarding the entire instrument. Likewise, in order to promote device sterility and economy, it is often useful to provide a re-usable handle portion with disposable and/or interchangeable end effectors, collectively known as a resposable device.

In a typical device wherein an actuation rod is proximally actuated to effect actuation of an end effector, a rod portion of the newly replaced end effector is connected to the actuation rod, and the outer shaft of the device upon which the end effector is to be assembled is attached to a casing portion of the end effector such that proper movement of the instrument occurs. Specifically, it is generally desirable that movement of the actuation rod within the outer shaft cause opening, closing, or other actuating movement of the end effector.

As it may be desirable to replace an end effector during a surgical procedure, it is most desirable that the disconnection of an end effector to be discarded and that the connection of a new end effector be a relatively simple and economical operation. At the same time, it is desirable that the connection be secure to avoid risk of the end effector becoming loose during use in a patient body and that the connection provide for effective actuation of the end effector. Thus, prior art devices have employed a variety of connections. For example, some devices—an example of which is shown as device 100 in FIG. 1A (the distal portion of which is shown schematically in magnified longitudinal cross-section)—use a threaded engagement 106 between the actuation rod 102 and the end effector rod 104 as well as a threaded engagement 114 between the outer shaft 110 and the end effector casing portion 112. Other devices—an example of which is shown as device 120 in FIG. 1A (the distal portion of which is shown in magnified longitudinal cross-section)—use a connection wherein the end effector casing 128 is threadedly connected to the device shaft 126, and the end effector rod 122 includes a ball or hemispherical proximal tip 124 that fits from the side into a keyhole aperture 132 of the actuation rod 130. It is preferable that the removal and replacement of an end effector require no special mechanical knowledge or capability on the part of the operator of the instrument, and that it may be done quickly and with a high degree of reliability, including in the low-light conditions commonly present in an operating suite during laparoscopic procedures.

In some of the arrangements set forth above, problems may arise in the initial engagement of the end effector with the actuation rod and shaft. For example, with a dual threaded connection such as illustrated in FIG. 1A, a user may experience difficulty in properly aligning and engaging both sets of threads in beginning the attachment of the new end effector to the instrument. As another example, with a side-mounted ball/socket inner connection such as illustrated in FIG. 1B (the distal portion of which is shown schematically in magnified longitudinal cross-section and rotated longitudinally 90° from the orientation of FIG. 1A), a user must spread the device handles to extend the actuation rod from the shaft, then align and place a ball structure on the end effector laterally into a side-opening keyhole aperture. Given the low-light conditions commonly present in a laparoscopy operating suite, this exacting operation can consume valuable time when replacing an end effector during a surgical procedure.

Each of the threaded and the ball/socket types of connection provide desirably secure mounting and functional capacity for laparoscopic instruments. However, previous attempts to combine these connection means require exacting lateral motion during engagement. It is therefore desirable to provide a laparoscopic instrument assembly having a disposable end effector wherein an end effector rod is disposed for axial movement within a tubular tip casing structure, with the end effector rod and casing structure, and the actuation rod and shaft, containing complementary ball/socket and threaded means for attachment, in which the attachment is simple in construction and economical to produce. It is also desirable to provide a laparoscopic instrument assembly of the type described wherein a replaceable end effector is easily and quickly assembled onto the instrument assembly in low-light conditions without requiring special instructions to the user, one aspect of which is providing for axial assembly (i.e., where an end effector is generally moved only within the longitudinal axis of the laparoscopic instrument rather than requiring, for example, lateral alignment of some components). Bayonet-type connections may also provide similar advantages for axial connection of both the inner (end effector rod to actuation rod) and outer (end effector casing to shaft) connections, and are therefore also be desirable.

### BRIEF SUMMARY

In one aspect, the present invention provides a laparoscopy device having an easy-to-engage mechanism for connecting a handle/shaft/actuation rod unit to an end effector. This mechanism may utilize an axially engageable inner ball/socket inner connection with a threaded outer connection, or an inner bayonet connection with either an outer bayonet connection or an outer threaded connection. In each of the embodiments, an outer portion of the end effector preferably is statically engaged with the shaft, and an inner portion of the end effector is engaged with the actuation rod such that axial movement of the actuation rod can actuate a tool end of the end effector. The present invention further relates to methods of use for the laparoscopy device.

In another aspect, the invention includes a surgical device. The surgical device includes a first handle member pivotably mounted to a second handle member, a generally tubular elongate shaft projecting distally from the second handle member, and an actuation rod disposed axially through the elongate shaft. The actuation rod is operatively connected near its proximal end to the first handle member, and a distal end portion of the actuation rod includes one of a ball member or a socketed prong assembly. The surgical device also includes an end effector assembly with an outer casing, an end effector rod disposed axially through the outer casing, and an actuatable tool end, with the end effector rod operably connected to the actuatable tool end. The outer casing is threadedly connectable to the elongate shaft and a proximal end portion of the end effector rod includes the other of a ball member or a socketed prong assembly which is complementary to and axially connectable to the opposite of a ball member or socketed prong assembly of the actuation rod.

In a further aspect, the present invention includes an end effector connection system. The end effector connection system includes a first surgical device component and a second surgical device component and is configured for axially connecting the first surgical device component to the second surgical device component. The first surgical device component includes a generally tubular first member having a first rod disposed axially therethrough. The first rod includes a socketed prong assembly with a plurality of prongs, at least one of which includes an indented socket. The generally tubular first member includes a first threaded surface. The second surgical device component includes a generally tubular second member having a second rod disposed axially therethrough. The second rod includes a ball structure, sized to be axially engagingly received between the plurality of prongs and at least partially into the indented socket, and the generally tubular second member includes a second threaded surface complementary to and engageable with the first threaded surface.

In yet another aspect, the invention includes an end effector connection system that includes a first surgical device component and a second surgical device component, with the system being configured for axially connecting the first surgical device component to the second surgical device component. The first surgical device component includes a generally tubular first member having a first rod disposed axially therethrough, and that first rod includes a male bayonet assembly comprising at least one boss extending from the first rod. The generally tubular first member also includes a first threaded surface. The second surgical device component includes a generally tubular second member having a second rod disposed axially therethrough. The second rod includes a female bayonet assembly, configured to axially engagingly receive the male bayonet assembly and the generally tubular second member includes a second threaded surface complementary to and engageable with the first threaded surface.

In still another aspect, the invention includes an end effector connection system configured for axially connecting a first surgical device component to a second surgical device component. The first surgical device component includes a generally tubular first member having a first rod disposed axially therethrough. The first rod includes a first male bayonet assembly comprising at least one boss extending laterally from the first rod, and the generally tubular first member includes a second male bayonet assembly comprising at least one boss extending laterally from the generally tubular first member. The second surgical device component includes a generally tubular second member having a second rod disposed axially therethrough. The second rod includes a first female bayonet assembly, configured to axially engagingly receive the first male bayonet assembly, and the generally tubular second member includes a second female bayonet assembly, which is configured to axially engagingly receive the second male bayonet assembly.

In still yet another aspect, the invention includes an end effector connection system comprising axially engageable first and second connecting means wherein the first connecting means includes an inner connecting means between an end effector rod and an actuation rod, and the second connecting means includes an outer connecting means between an end effector casing and an outer shaft. In order to lockingly engage the end effector rod with the actuation rod, the first connecting means requires no rotation greater than 360°.

In another aspect, the invention includes an end effector connection system that includes axially engageable first and second inner connecting members and axially engageable first and second outer connecting members. The first and second inner connecting members are generally axially disposed in the first and second outer connecting members, respectively. The first inner connecting member includes a generally cylindrical body having a laterally protruding portion disposed near its distal end, and the second inner connecting member has a receiving aperture configured to axially receive the first inner connecting member.

### BRIEF DESCRIPTION OF THE DRAWINGS

FIGS. 1A and 1B illustrate a illustrate prior art end effector connection systems;

FIG. 2A illustrates a side view with partial cross-section of a surgical device of the present invention, disassembled;

FIGS. 2B depicts a side view with partial cross-section of a surgical device of the present invention, assembled;

FIG. 2C shows a longitudinal section view of an end effector embodiment of the present invention, with connecting means therefor;

FIG. 2D illustrates a perspective view of an end effector embodiment of the present invention, with connecting means therefor;

FIG. 2E shows a schematic view of ball/socket connecting means for an end effector of the present invention,

FIGS. 3A-3E depict a method of using an end effector connection system of the present invention;

FIGS. 4A-4F illustrate schematically the components of another end effector connection system of the present invention; and

FIGS. 5A-5C show schematically components of yet another end effector connection system of the present invention.

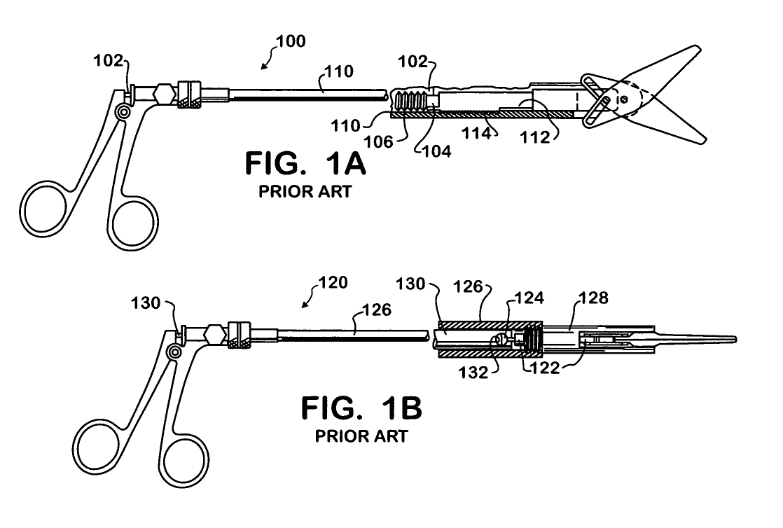

### DETAILED DESCRIPTION

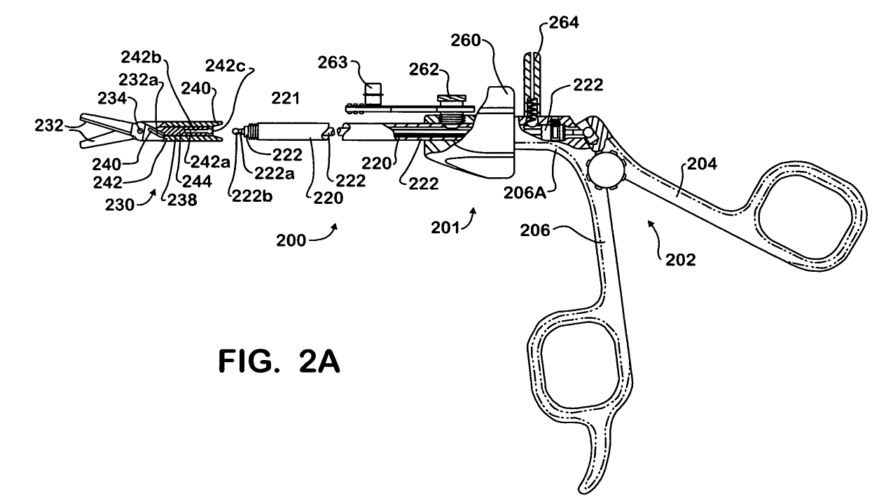

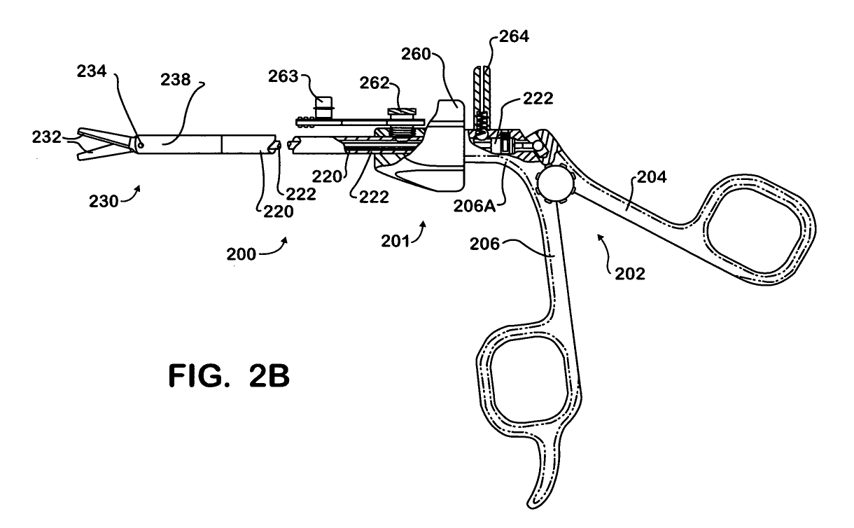

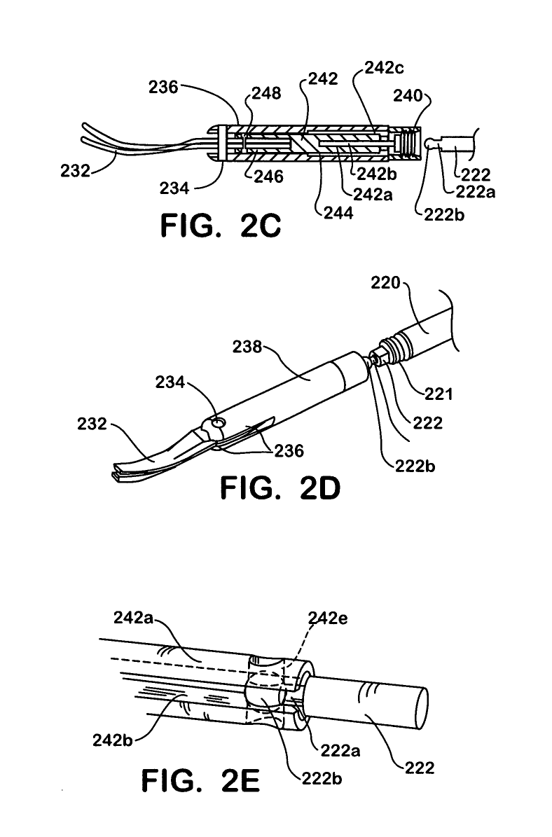

A first embodiment of a laparoscopy device 200 is illustrated with reference to FIGS. 2A-2E. FIG. 2A shows a side view with partial cross-section of the disassembled device, FIG. 2B shows the same side view with partial cross-section of the assembled device, and FIG. 2D shows a partial perspective view of the distal end of the device. As illustrated in FIGS. 2A-2E, the laparoscopy device includes two main components: a main body 201 and an end effector 230. FIGS. 2C and 2E show detail views of the connection between the main body 201 and the end effector 230. In a preferred embodiment, the main body 201 is reusable, and the end effector 230 is disposable and replaceable. The main body 201 includes a handle 202 and a shaft 220 extending distally therefrom. The handle 202 includes a thumb ring member 204 pivotably attached at a pivot pin 208 to a finger ring member 206, which includes an upper barrel portion 206a. The elongate tubular shaft 220 extends distally from the upper barrel portion 206a of the finger ring member 206. An actuation rod 222 extends distally from an upper portion of the thumb ring member 204 through the shaft 220. This configuration provides for reciprocating axial movement of the actuation rod 222 in the shaft 220 upon pivoting the thumb ring member 204 relative to the finger ring member 206. (Those of skill in the art will appreciate that the handle may be configured such that the thumb ring handle is static with a barrel portion attached to a shaft, and that the finger ring handle be pivotable relative to the thumb ring handle.) At the distal end of the shaft 220, the end effector 230 is operably connected both to the shaft 220 and the actuation rod 222. The distal end of the main body shaft 220 includes an outer threaded surface 221 that is complementary to the inner threaded surface 240 of the end effector casing 238. The distal end portion of the actuation rod 222 includes a narrow portion 222a and a distal ball member 222b.

In the preferred embodiment illustrated in FIGS. 2A-2E, the end effector 230 includes two cutting blade members 232 mounted pivotably on a pivot pin 234. In other embodiments, one of the blade members may be fixed, or both may be replaced with, for example, forceps, graspers, needle holders, clamps, dissectors, and spreaders, or another actuatable tool end. The pivot pin 234 is mounted between distal arms 236 of a generally cylindrical end effector casing 238. The blade members 232 each include a proximal camming aperture 232a. The proximal end of the end effector casing 238 includes an inner threaded surface 240. In a preferred embodiment, the end effector casing 238 includes an electro-insulative outer coating that forms a seal with an electro-insulative outer coating of the main body shaft 220. A generally cylindrical end effector rod 242 is mounted in a longitudinal central cavity 244 of the end effector casing 238.

The distal end portion of the end effector rod 242 includes a pair of forks 246, between which is disposed a camming pin 248. In the illustrated embodiment, the camming pin 248 is disposed through the camming apertures 232a of the blade members 232 such that distal axial movement of the end effector rod 242 cams the blade members 232 such that their distal ends move open/apart from each other, and proximal axial movement of the end effector rod 242 cams the blade members 232 such that their distal ends move closed/toward each other. The proximal end portion of the end effector rod 242 includes a socketed prong assembly having a pair of generally semi-cylindrical, proximally-extending prongs 242a biased toward each other. At least one of the generally flat inner prong surfaces 242b include one or more generally matching indentations or openings 242c that effectively form a socket for receiving the distal ball member 222b of the actuation rod 222. In a preferred embodiment shown most clearly in FIGS. 2C and 2E, a socket is provided by a generally cylindrical aperture 242e disposed perpendicular to a plane between the prongs 242a and disposed adjacent their distal ends. The intersection of the aperture 242e with the inner prong surfaces 242b provides a matched pair of ball-receiving openings 242c that form a socket for receiving the distal ball member 222b of the actuation rod 222. In alternative embodiments (e.g., those embodiments with a similar or different ball member geometry), the openings 242c may be, for example, generally semi-cylindrical or hemi-spherical. In the embodiment shown in FIG. 2E, the proximal ends of the generally flat inner prong surfaces 242b each also include a depression 242d configured to aid passage of the distal ball member 222b of the actuation rod 222 into engagement with the openings 242c. The ball member 222b may have different geometries in different embodiments within the present invention such that it may be, for example, generally spherical, generally cylindrical, or frustoconical. Also, in other embodiments within the scope of the present invention, the socketed prong assembly may include three or more prongs, at least one of which has an indentation for engaging and retaining the ball member.

Together, the end effector 230 and the main body 201 include an end effector connection system. In other words, the end effector 230 is operably connectable to the main body 201 using a connecting means of the present invention as described below. When the end effector 230 is engaged with the main body 201, the inner threaded surface 240 of the end effector casing 238 is threadedly engaged with the outer threaded surface 221 of the shaft 220, and the distal ball member 222b of the actuation rod 222 is in a “snap-fit” engagement with the depressions 242d of the end effector proximal prongs 242. However, those of skill in the art will appreciate that, in alternative embodiments within the scope of the present invention, the relative position of the ball and prongs may be reversed such that a proximal ball member on the end effector rod may be engagingly received by distal prongs on the actuation rod.

The illustrated embodiment depicts the handle members 204, 206 opening in scissors-like fashion to actuate the end effector 230. Specifically, when the handle members 204, 206 are together, the actuation rod 222 is retracted proximally and the blade members 232 are together in a closed state. And, as shown in FIG. 2A, when the handle members 204, 206 are spread apart, the actuation rod 222 is extended distally and the blade members 232 are spread apart in an open state. This configuration allows the blade members 232 actively to be controlled for an opening motion (e.g., such as for spreading tissue) and for a closing motion (e.g., such as for grasping or cutting tissue).

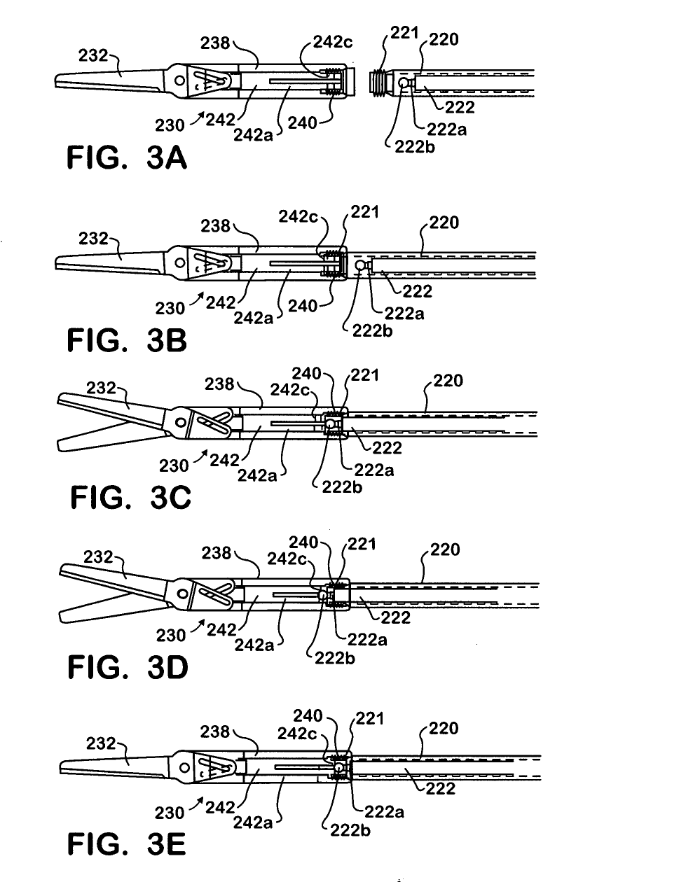

The above-described embodiment preferably is configured for ease of engagement by use of the following method, which is able to be conducted in low-light conditions, and which is described with reference to FIGS. 3A-3E. First, as shown in FIG. 3A-3B, with the device handle members 204, 206 (not shown) drawn together and the actuation rod 222 retracted accordingly, a user places the proximal end of the end effector casing 238 over the distal end of the main body shaft 220 and twists the end effector 220 to engage the inner end effector threads 240 with the outer shaft threads 221. (Those of skill in the art will appreciate that, in other embodiments, the inner threads may be on the end effector, and the outer threads may be on the shaft.) Next, as shown in FIGS. 3C-3D, the user spreads the handle members 204, 206 (not shown), which moves the actuation rod 222 distally such that the distal ball member 222b is forced axially between the prongs 242a of the end effector rod 242 until it is engaged by the openings 242c and a proximal portion of the prongs 242a biasedly closes about the narrow portion 222a of the actuation rod 222. In a preferred embodiment, during the transition shown from FIG. 3C, to FIG. 3D, the user will have a tactile sensation of the snap-fit engagement of the ball member 222b with the socket formed by the depressions 242d of prongs 242a, and may also hear an audible click of engagement. Thereafter, as shown in FIG. 3E, the interface of ball member 222b with prongs 242a maintains an operative engagement between the main body 201 and the end effector 230. Specifically, the proximal ends of prongs 242a are captured by the main body shaft 220, the end effector casing 238 is held generally static relative to the main body shaft 220, and the end effector rod 242 is operatively connected to the actuation rod 222 such that actuation of the handles 204, 206 transmits axial movement through the actuation rod 222 to the end effector 230 to open and close the end effector blade members 232 while the end effector 230 is secured to the main body shaft 220 and actuation rod 222.

In an alternative method, the actuation rod ball member 222b may first be axially engaged with the end effector prongs 242a, and then the end effector casing 238 may be threadedly engaged with the main body shaft 220.

In preferred embodiments of the present device (such as illustrated in FIGS. 2A-2E), the shaft 220 includes indexed rotating means such as a rotation knob 260 with corresponding internal structure of a type known in the art, so that it can be rotated about its (220) longitudinal axis in an indexed fashion. The shaft 220 also preferably includes means for flushing its internal portion, such as a Luer port 262 with a cap 263. Preferred embodiments of the present device 200 are configured for use with monopolar-type electrosurgical apparatus such that the exterior of the main body 201 and the end effector casing 238 are insulated, and an electrode such as a Bovie post 264 is provided to direct current from an electrosurgical generator (not shown) through the actuation rod to the tool end (e.g., blade members 232) where it may be used, for example, in cutting and coagulation. Those of skill in the art will also appreciate that the “ball/socket plus threaded” end effector connection system described above is also useful within the scope of the present invention for connecting a first and second surgical device component in a non-laparoscopic tool device.

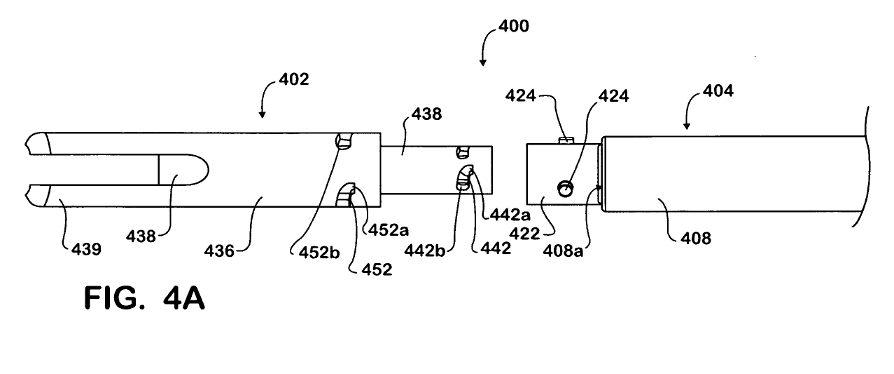

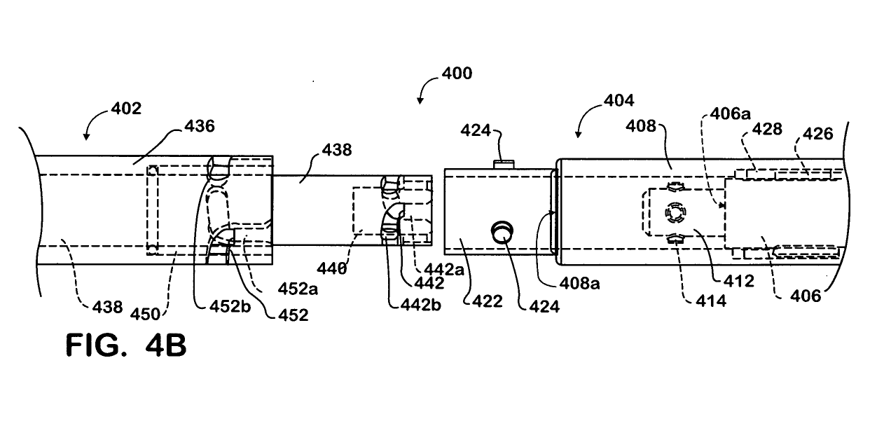

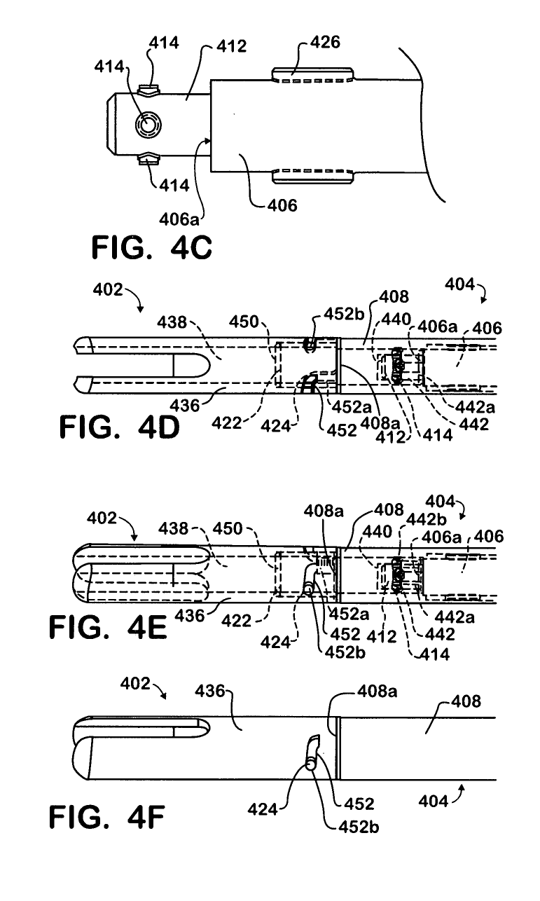

FIGS. 4A-4C depict another end effector connector system 400 embodiment of the present invention. As shown in these figures, this embodiment of an end effector connection system includes an inner bayonet connection and an outer bayonet connection for connecting an end effector 402 to the main body 404 of a surgical instrument of the type shown in FIG. 2A.

The proximal portion of the instrument main body 404, including its handle, is not shown. The main body 404 includes an actuation rod 406 disposed axially through an outer shaft 408. A distal portion of the outer shaft 408 includes a male bayonet structure with a distal outer shaft portion 422 having an outer diameter less than that of the major length of the outer shaft 408. The border of the distal portion 422 with the larger outer diameter of the outer shaft 408 is defined by a lip 408a, the surface of which is in a plane generally perpendicular to the longitudinal axis. A plurality of outer shaft bosses 424 extends radially from that distal outer shaft portion 422. The illustrated embodiment includes four outer shaft bosses 424, but those of skill in the art will appreciate that more or fewer bosses may be used in other embodiments. Although it is preferable to have a plurality of bosses, a single outer shaft boss may be used within the scope of the present invention. As is shown most clearly in FIG. 4C, a distal portion of the actuation rod 406 includes a male bayonet structure with a distal actuation rod portion 412 having an outer diameter less than that of the major length of the actuation rod 406. The border of the distal actuation rod portion 412 with the larger outer diameter of the actuation rod 406 is defined by a lip 406a, the surface of which is in a plane generally perpendicular to the longitudinal axis. A plurality of actuation rod bosses 414 extends radially from that distal actuation rod portion 412, and preferably is radially offset relative to the plurality of outer shaft bosses 424. The illustrated embodiment includes four actuation rod bosses 414, but those of skill in the art will appreciate that more or fewer bosses may be used in other embodiments. Although it is preferable to have a plurality of bosses, a single actuation rod boss may be used within the scope of the present invention.

The end effector 402 includes an outer casing 436. A generally cylindrical end effector rod 438 is disposed in the outer casing 436 in a manner allowing axial (but not rotational) sliding movement to actuate a tool end in the same manner as described with reference to the embodiment of FIG. 2A, or in another manner known to those of skill in the art. The tool end (e.g., blade members) is not shown, but a forked distal end 439 is provided for mounting the tool end. The proximal portion of the end effector rod 438 includes a female bayonet structure, which includes an inner receiving channel 440 and a plurality of curved, L-shaped boss-receiving slots 442. The number of boss-receiving slots 442 preferably is the same as the number of bosses 414 on the actuation rod 406. The proximal portion of each receiving slot is a groove 442a on the inner surface of the inner receiving channel 440. The groove 442a preferably is generally parallel to the central longitudinal axis of the end effector 402. The distal portion 442b of each slot 442 curves radially with the outer contour of the end effector rod 438, and preferably is nearly perpendicular to the proximal slot portion 442a, but is slightly angled toward the distal end of the end effector 402. In the illustrated embodiment, the distal slot portion 442b is open to the exterior of the end effector rod 438.

The outer casing 436 includes a female bayonet structure, which includes a generally cylindrical shaft-receiving channel 450 and a plurality of curved, L-shaped boss-receiving slots 452. The number of boss-receiving slots 452 preferably is the same as the number of outer shaft bosses 424 on the outer shaft 408. The proximal portion of each boss-receiving slot 452 is a groove 452a on the inner surface of the generally cylindrical shaft-receiving channel 450. The groove 452a preferably is generally parallel to the central longitudinal axis of the end effector 402. The distal portion 452b of each slot 452 curves radially with the outer contour of the outer casing 436, and preferably is nearly perpendicular to the proximal slot portion 452a, but is slightly angled toward the distal end of the end effector 402. In the illustrated embodiment, the distal slot portion 452b is open to the exterior of the end effector rod 438. This open portion allows for visual verification of full engagement, as shown in FIG. 4F.

The actuation rod 406 preferably includes a longitudinal fin 426 projecting radially therefrom and engaging a complementarily-sized fin-receiving groove 428 on the inner surface of the outer shaft 408 in a manner allowing axial, but not rotational movement of the actuation rod 406 relative to the outer shaft 408. In this manner, the rotational orientation of the actuation rod bosses 414 and the outer shaft bosses 424 is held consistently so that the female bayonet structures of the end effector 402 may readily be axially engaged with the male bayonet structures of the main body 404. This configuration allows the outer shaft 408 and the actuation rod 406 to be rotated together, and permits axial actuation of the actuation rod 406 for transmitting movement to the tool end (not shown).

A method of engaging the double bayonet end effector connection system 400 is described with reference to FIGS. 4A-4C. To engage the end effector 402 to the main body 404, a user axially inserts the proximal portion of the end effector rod 438 into the distal end of the main body's outer shaft 408. The user then rotates the end effector 402 relative to the outer shaft 408 (or vice versa) to align the outer boss-receiving slots 452 of the end effector outer casing 436 with the outer shaft bosses 424 of the outer shaft 408. This action simultaneously aligns the female bayonet member of the end effector 402 with the male bayonet member of the actuation rod 406. As shown in FIG. 4D, once aligned, the user moves the end effector 402 and the main body 404 axially together such that the distal end of the actuation rod 406 enters the end effector rod's inner receiving channel 440, the outer shaft bosses 424 slide into the proximal groove portion 452a of each boss-receiving slot 452 of the end effector outer casing 436, and the actuation rod bosses 414 slide into the proximal groove portion 442a of each boss-receiving slot 442 of the end effector rod 438. Next, as shown in FIG. 4E, the user rotates the end effector 402 counterclockwise relative to the outer shaft 408 (or vice versa). This rotation engages the main body bosses 414, 424 into the respective distal slot portions 442b, 452b of the end effector. Because each of the distal slot portions 442b, 452b of the end effector is slightly distally angled, this boss-slot engagement forms a frictional lock as the proximal end of the end effector rod 438 is forced against the actuation rod lip 406a and the proximal end of the end effector outer casing is forced against the outer shaft lip 408a. Each of the lips 406a, 408a may alternatively include a surface-mounted gasket (not shown) that is compressed and that helps to provide a frictional lock. As shown in the illustrated embodiment, this engagement method requires only a small rotational motion after the initial axial engagement. In the illustrated embodiment, the rotation required to lock in the engagement is less than 900. In other embodiments, including those with more or fewer bosses, the rotation required is less than 360°.

For the end effector system of the surgical device embodiment illustrated in FIGS. 2A-2E, engaging the inner connecting means (i.e., those means connecting the end effector rod with the actuation rod) does not require any rotation of either the end effector or the main body. Other connection systems including means that do require rotation to engage the inner connecting means are disclosed below, but such embodiments require rotation of less than 360°, preferably less than 180°, and most preferably less than 90°. All such connection systems provide the advantage of minimizing the amount of movement a user must employ to securely engage an end effector to a main body in a surgical device, even when some rotation (e.g., for a bayonet or threaded connection) is required to secure the outer shaft to the end effector casing. Those of skill in the art will appreciate that the ball/socket inner connection means described with reference to FIGS. 2A-2E may be used with an outer bayonet connector as described with reference to FIGS. 4A-4C.

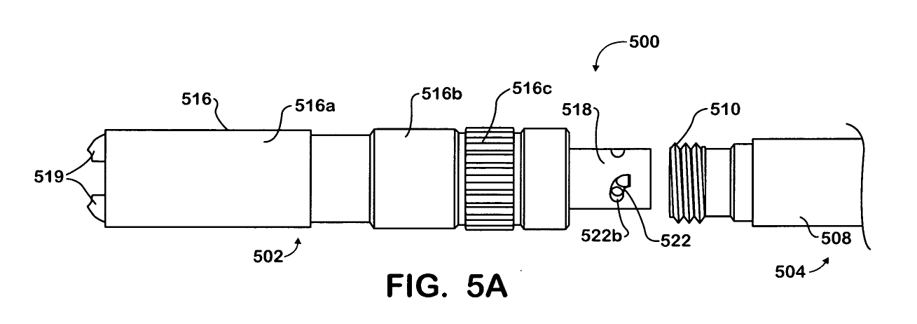

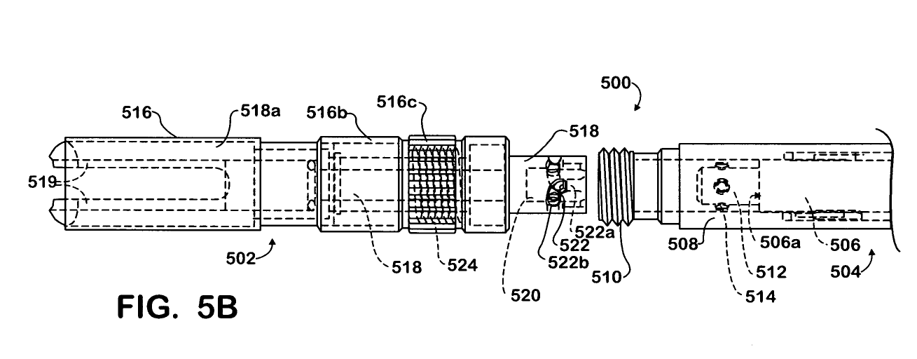

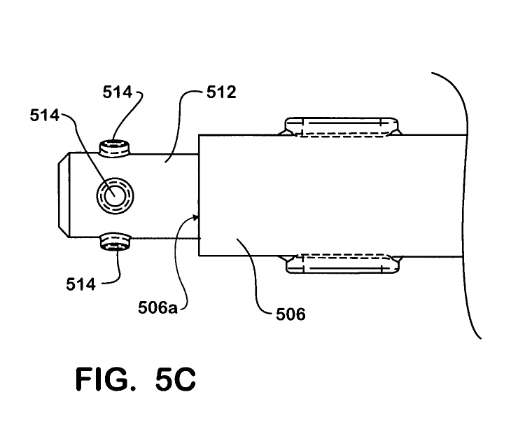

FIGS. 5A-5C illustrate another end effector connection system 500 embodiment of the present invention. As shown in these figures, this embodiment of an end effector connection system includes an inner bayonet connection and an outer threaded connection for connecting an end effector 502 to the main body 504 of a surgical instrument of the type shown in FIG. 2A.

The proximal portion of the instrument main body 504, including its handle, is not shown. The main body 504 includes an actuation rod 506 and an outer shaft 508. A distal portion of the outer shaft 508 includes an outer threaded surface 510. As is shown in FIGS. 5B-5C, a distal portion of the actuation rod 506 includes a male bayonet structure including a distal actuation rod portion 512 having an outer diameter less than that of the major length of the actuation rod 506. The border of the distal actuation rod portion 512 with the larger outer diameter of the actuation rod 506 is defined by a lip 506a, the surface of which is in a plane generally perpendicular to the longitudinal axis. A plurality of bosses 514 extends radially from that distal actuation rod portion 512. The illustrated embodiment includes four bosses 514, but those of skill in the art will appreciate that more or fewer bosses may be used in other embodiments. Although it is preferable to have a plurality of bosses, a single boss may be used within the scope of the present invention.

The end effector 502 includes an outer casing 516. A generally cylindrical end effector rod 518 is disposed in the outer casing 516 in a manner allowing axial sliding movement to actuate a tool end in the same manner as described with reference to the embodiment of FIG. 2A, or in another manner known to those of skill in the art. The tool end (e.g., blade members) is not shown, but a forked distal end 519 is provided for mounting the tool end. The proximal portion of the end effector rod 518 includes a female bayonet structure, which includes an inner receiving channel 520 and a plurality of curved, L-shaped boss-receiving slots 522. The number of boss-receiving slots 522 preferably is the same as the number of bosses 514 on the actuation rod 506. The proximal portion of each receiving slot is a groove 522a on the surface of the inner receiving channel 520. The groove 522a preferably is generally parallel to the central longitudinal axis of the end effector 502. The distal portion 522b of each slot 522 curves radially with the outer contour of the end effector rod 518, and preferably is nearly perpendicular to the proximal slot portion 522a, but is slightly angled toward the distal end of the end effector 502. In the illustrated embodiment, the distal slot portion 522b is open to the exterior of the end effector rod 518.

The end effector outer casing 516 includes two portions. A distal casing portion 516a is attached to the forked distal end 519 in a manner allowing axial movement as described above. A proximal casing portion 516b is rotatably connected to the distal casing portion 516a such that it can be rotated about the central longitudinal axis of the end effector 502 without moving significantly axially. The proximal casing portion 516b includes an inner threaded surface 524 that is complementary to the outer threaded surface 510 of the main body's outer shaft 508. And, the proximal casing portion 516b preferably includes a textured outer surface 516c that provides enhanced friction for gripping the proximal casing portion 516b and rotating it (e.g., to engage the inner threaded surface 524 with the outer threaded surface 510 of the main body's outer shaft 508).

A method of engaging the threaded-plus-bayonet end effector connection system 500 is described with reference to FIGS. 5A-5C. To engage the end effector 502 to the main body 504, a user axially inserts the proximal portion of the end effector rod 538 into the distal end of the main body's outer shaft 508. The user then rotates the end effector 502 relative to the outer shaft 508 (or vice versa) to align the boss-receiving slots 522 of the end effector rod 518 with the bosses 514 of the actuation rod 506. Once aligned, the user moves the end effector 502 and the main body 504 axially together such that the distal end of the actuation rod 506 enters the end effector rod's inner receiving channel 520, the actuation rod bosses 514 slide into the proximal groove portion 522a of each boss-receiving slot 522 of the end effector rod 518. Next, the user rotates the end effector 402 counterclockwise relative to the outer shaft 408 (or vice versa). This rotation engages the bosses 514 into the distal slot portions 522b of the end effector rod 518. Because each of the distal slot portions 522b of the end effector is slightly distally angled, this boss-slot engagement forms a frictional lock as the proximal end of the end effector rod 518 is forced against the actuation rod lip 506a. As shown in the illustrated embodiment, this engagement method requires only a small rotational motion after the initial axial engagement. In the illustrated embodiment, the rotation required to lock in the engagement is less than 90°. In other embodiments, including those with more or fewer bosses, the rotation required is less than 360°. Finally, the user rotates the proximal casing portion 516b to engage its inner threaded surface 524 with the outer threaded surface 510 of the main body's outer shaft 508. Those of skill in the art will appreciate that the male and female bayonet portions described herein may be reversed in orientation in other embodiments, within the scope of the present invention.

Those of skill in the art will also appreciate that other variants of the surgical device and end effector connector system embodiments described herein may also be practiced within the scope of the present application. It is therefore intended that the foregoing detailed description be regarded as illustrative rather than limiting. It should be understood that the following claims, including all equivalents, are intended to define the spirit and scope of this invention.

We claim:

1. A surgical device, comprising:
a first handle member pivotably mounted to a second handle member;
a generally tubular elongate shaft projecting distally from the second handle member, the elongate shaft comprising a distal threaded surface;
an actuation rod disposed axially through the elongate shaft, the actuation rod operatively connected near its proximal end to the first handle member, and a distal end portion of the actuation rod comprising one of a ball member or a socketed prong assembly; and
an end effector assembly comprising
an outer casing having a proximal threaded surface complementary to the distal threaded surface of the elongate shaft,
an end effector rod disposed axially through the outer casing, and
an actuatable tool end, the end effector rod operably connected to the actuatable tool end;
wherein a proximal end portion of the end effector rod includes the other of a ball member or a socketed prong assembly which is complementary to and axially connectable with the opposite of a ball member or socketed prong assembly of the actuation rod.

2. The surgical device of claim 1, wherein the distal end portion of the actuation rod comprises a ball member, and the proximal end portion of the end effector comprises a socketed prong assembly.

3. The surgical device of claim 1, wherein the distal end portion of the actuation rod comprises a socketed prong assembly, and the proximal end portion of the end effector comprises a ball member.

4. The surgical device of claim 1, wherein the actuation rod and the actuatable tool end each comprise an electroconductive material.

5. The surgical device of claim 1, further comprising an electroinsulative covering disposed on at least an external surface portion of the device.

6. The surgical device of claim 1, wherein the actuatable tool end is selected from a group consisting of cutting blade members, forceps, graspers, needle holders, clamps, dissectors, and spreaders.

7. The surgical device of claim 1, wherein the socketed prong assembly comprises two prongs biased toward each other, each prong including a ball-receiving opening disposed adjacent an end thereof.

8. The surgical device of claim 1, wherein the elongate shaft is configured to be rotated about its central longitudinal axis relative to the second handle member.

9. A method for assembling the surgical device of claim 1, the method comprising:
providing the surgical device of claim 1;
threadedly engaging the outer casing to the elongate shaft;
actuating the first handle member in a manner extending the actuation rod distally such that the one of a ball member or a socketed prong assembly axially engages the other of a ball member or socketed prong assembly.

10. An end effector connection system comprising a first surgical device component and a second surgical device component, the connection system being configured for axially connecting the first surgical device component to the second surgical device component,
the first surgical device component comprising
a generally tubular first member having a first rod disposed axially therethrough;
wherein the first rod includes a socketed prong assembly comprising a plurality of prongs, at least one of which includes an indented socket; and
wherein the generally tubular first member includes a first threaded surface;
the second surgical device component comprising
a generally tubular second member having a second rod disposed axially therethrough;
wherein the second rod includes a ball structure, sized to be axially engagingly received between the plurality of prongs and at least partially into the indented socket; and
wherein the generally tubular second member includes a second threaded surface complementary to and engageable with the first threaded surface.

11. The end effector connector system of claim 10, wherein the first surgical device component comprises a main body of a laparoscopic tool device, said main body comprising an actuatable handle assembly; said generally tubular first member comprising an outer shaft projecting distally from the handle assembly and said first rod operably connected to the actuatable handle assembly in a manner configured to provide for reciprocating axial movement of the first rod in the outer shaft upon actuation of the handle assembly.

12. The end effector connector system of claim 10, wherein the first surgical device component comprises an end effector assembly, said end effector assembly comprising at least one actuatable end effector member operably connected to the first rod.

13. The end effector connector system of claim 10, wherein the second surgical device component comprises a main body of a laparoscopic tool device, said main body comprising a handle assembly and said generally tubular second member comprising an outer shaft projecting distally from the handle assembly.

14. An end effector connection system comprising a first surgical device component and a second surgical device component, the connection system being configured for axially connecting the first surgical device component to the second surgical device component,
the first surgical device component comprising
a generally tubular first member having a first rod disposed axially therethrough;
wherein the first rod includes a male bayonet assembly comprising at least one boss extending from the first rod; and
wherein the generally tubular first member includes a first threaded surface;
the second surgical device component comprising
a generally tubular second member having a second rod disposed axially therethrough;
wherein the second rod includes a female bayonet assembly, configured to axially engagingly receive the male bayonet assembly; and
wherein the generally tubular second member includes a second threaded surface complementary to and engageable with the first threaded surface.

15. An end effector connection system comprising a first surgical device component and a second surgical device component, the connection system being configured for axially connecting the first surgical device component to the second surgical device component,
the first surgical device component comprising
a generally tubular first member having a first rod disposed axially therethrough;
wherein the first rod includes a first male bayonet assembly comprising at least one boss extending laterally from the first rod; and
wherein the generally tubular first member includes a second male bayonet assembly comprising at least one boss extending laterally from the generally tubular first member;
the second surgical device component comprising
a generally tubular second member having a second rod disposed axially therethrough;
wherein the second rod includes a first female bayonet assembly, configured to axially engagingly receive the first male bayonet assembly; and
wherein the generally tubular second member includes a second female bayonet assembly, configured to axially engagingly receive the second male bayonet assembly.

16. An end effector connection system comprising axially engageable first and second connecting means,
wherein the first connecting means comprises an inner connecting means between an end effector rod and an actuation rod and the second connecting means comprises an outer connecting means between an end effector casing and an outer shaft;
said first connecting means requiring no rotation greater than 360° to lockingly engage the end effector rod with the actuation rod.

17. The end effector connection system of claim 16, wherein a rotation of less than 180° is required to lockingly engage the end effector rod with the actuation rod.

18. The end effector connection system of claim 17, wherein the second connecting means is selected from one of a threaded connecting means and a bayonet connecting means.

19. The end effector connection system of claim 16, wherein a rotation of less than 90° is required to lockingly engage the end effector rod with the actuation rod.

20. The end effector connection system of claim 19, wherein the second connecting means is selected from one of a threaded connecting means and a bayonet connecting means.

21. The end effector connection system of claim 16, wherein the first connecting means comprises a ball member disposed adjacent a distal end portion of the actuation rod and a socketed prong assembly disposed adjacent a proximal end portion of the end effector rod, said socketed prong assembly configured to engagingly receive the ball member.

22. The end effector connection system of claim 21, wherein the second connecting means is selected from one of a threaded connecting means and a bayonet connecting means.

23. The end effector connection system of claim 16, wherein the first connecting means comprises a ball member disposed adjacent a proximal end portion of the end effector rod and a socketed prong assembly disposed adjacent a distal end portion of the actuation rod, said socketed prong assembly configured to engagingly receive the ball member.

24. The end effector connection system of claim 23, wherein the second connecting means is selected from one of a threaded connecting means and a bayonet connecting means.

25. The end effector connection system of claim 16, wherein the first connecting means comprises a female bayonet assembly disposed adjacent a proximal end portion of the end effector rod and a male bayonet assembly disposed adjacent a distal end portion of the actuation rod, said female bayonet assembly configured to engagingly receive the male bayonet assembly.

26. The end effector connection system of claim 25, wherein the second connecting means is selected from one of a threaded connecting means and a bayonet connecting means.

27. The end effector connection system of claim 16, wherein the first connecting means comprises a male bayonet assembly disposed adjacent a proximal end portion of the end effector rod and a female bayonet assembly disposed adjacent a distal end portion of the actuation rod, said female bayonet assembly configured to engagingly receive the male bayonet assembly.

28. The end effector connection system of claim 27, wherein the second connecting means is selected from one of a threaded connecting means and a bayonet connecting means.

30. An end effector connection system comprising:
Axially engageable first and second inner connecting members and axially engageable first and second outer connecting members, wherein the first and second inner connecting members are generally axially disposed in the first and second outer connecting members, respectively;
Said first inner connecting member comprising a generally cylindrical body having a laterally protruding portion disposed near its distal end and said second inner connecting member having a receiving aperture configured to axially receive the first inner connecting member.

31. The end effector connection system of claim 30, wherein the first outer connecting member is a male bayonet member and the second outer connecting member is a female bayonet member configured to engagingly receive the male bayonet member.

32. The end effector connection system of claim 30, wherein the first outer connecting member comprises a first threaded surface and the second outer connecting member comprises a second threaded surface that is configured to engage the first threaded surface.

33. An end effector for a surgical device, the end effector comprising:
an outer casing comprising a threaded surface;
a distal tool end; and
an end effector rod disposed at least partially through the outer casing, wherein a proximal end of the end effector rod comprises a connecting member that is selected from one of a male bayonet member, a female bayonet member, a ball member, and a socket member, and wherein the connecting member is configured to be axially connected to a surgical device.
